(19) United States
(12) Patent Application Publication
(10) Pub. No.: US 2009/0148833 A1
(43) Pub. Date: Jun. 11, 2009

(54) DEVICES FOR GENERATING DETECTABLE POLYMERS

(75) Inventor: Eric K. Engelhard, Davis, CA (US)

Correspondence Address: FISH & RICHARDSON P.C., PO BOX 1022, MINNEAPOLIS, MN, 55440-1022, US

(73) Assignee: FAIR ISAAC CORPORATION, Minneapolis, MN (US)

(21) Appl. No.: 11/690,400

(22) Filed: Mar. 23, 2007

(51) Int. Cl. C12Q 1/68; C12M 1/34

(52) U.S. Cl. 435/6; 435/287.2

(57) ABSTRACT

This document provides systems, devices, and methods involved in generating detectable polymers. For example, diagnostic systems, diagnostic devices, primer systems, and collections of primer systems are provided.

### BACKGROUND

1. Technical Field

This document relates to systems, devices, and methods involved in generating detectable polymers.

2. Background Information

Many different types of devices exist for generating polymers such as labeled deoxyribonucleic acids. For example, tubes, tube retainer trays, microtiter plates, microfluidic cards, and glass slides containing arrays have been fabricated to allow a user to generate polymers. The HT7900 Micro Fluidic Card™ is an example of a microfluidic card designed to allow a user to generate polymers. In this case, the microfluidic card functions as a structured array of reaction chambers and contains input ports for inserting samples into the card. The HT7900 Micro Fluidic Card™ is available from Applied Biosystems Group (Foster City, Calif.).

In addition, many different techniques have been developed to detect a generated polymer. For example, machines designed to read fluorescent signals from each well of a microtiter plate have been developed. The FLx800™ reader is an example of an absorbance and fluorescence instrument for measuring samples in various microplate arrangements. The reader can be used in numerous fluorescence and absorbance applications in research and routine investigations. Its fluorescence filters are arranged in filter wheels. The reader can handle 6, 48, 96, and 384 well plates and can detect wavelengths in the fluorescence spectral range. Gen5™ data collection and analysis software can be used for data capture, and standard reads and data can be downloaded into Excel™ for further analysis. Dual optical channels can allow for measurements from above or below the plate. Light to and from the samples can be focused by a lens. The FLx800™ reader is available from BioTek Instruments, Inc. (Winooski, Vt.).

### SUMMARY

This document relates to systems, devices, and methods involved in generating detectable polymers. For example, this document provides diagnostic systems, diagnostic devices, primer systems, and collections of primer systems. A diagnostic system can include a diagnostic device containing a collection of primer systems. This document also provides methods for making diagnostic systems, diagnostic devices, primer systems, and collections of primer systems. For example, this document provides methods for making a diagnostic device containing a collection of primer systems. The systems, devices, and methods provided herein can be used to generate detectable polymers such as amplified deoxyribonucleic acid molecules. In addition, the systems, devices, and methods provided herein can be used to detect the presence or absence of nucleic acid encoding a Staphylococcus coagulase, hyaluronidase, mecA, or TSST polypeptide, or a portion thereof, within samples. Detecting the presence or absence of such nucleic acids can help clinicians provide important diagnostic and prognostic information to patients.

The description provided herein is based, in part, on the discovery of effective primer systems for generating detectable polymers. For example, a diagnostic device provided herein can contain primer systems effective to detect the presence or absence of nucleic acid encoding a Staphylococcus coagulase, hyaluronidase, mecA, or TSST polypeptide within samples. Such a diagnostic device can be used to aid clinicians in assessing a patient's prognosis. The description provided herein also is based, in part, on the discovery of primer systems having the ability to not only amplify particular nucleic acid sequences from different Staphylococcus bacteria, but also to not amplify nucleic acid sequences from non-Staphylococcus bacterial sources such as a human's genome. In addition, the description provided herein is based, in part, on the discovery of primer systems that can be used simultaneously with a collection of primer pairs under the same amplification reaction conditions to amplify different target nucleic acids if present in the sample being tested.

In general, one aspect of this document features a device comprising, or consisting essentially of, a housing having a plurality of locations, wherein each of the locations contains a primer system, wherein the primers of each primer system are between 18 and 28 nucleotides in length and have a theoretical melting temperature between 58° C. and 62° C., wherein the device comprises at least one primer system capable of producing an amplification product diagnostic for nucleic acid encoding a Staphylococcus coagulase, hyaluronidase, mecA, or TSST polypeptide, and wherein each amplification product, when produced, is between 100 and 400 nucleotides in length. Each of the locations can be a chamber. Each of the locations can be a well. The primers of each primer system can be between 23 and 27 nucleotides in length. The primers of each primer system can have a theoretical melting temperature between 59° C. and 61° C. The housing can comprise additional locations, wherein each of the additional locations contains a primer pair. At least one of the additional locations can comprise a primer pair capable of producing an amplification product from human nucleic acid. Each of the locations can comprise an intercalating dye, and wherein each amplification product, when produced, can be labeled with the intercalating dye. The intercalating dye can be a green fluorescent dye. The intercalating dye can be SYBR Green, LC Green, or SYTO9. Each amplification product, when produced, can be between 100 and 300 nucleotides in length.

In another aspect, this document features method for detecting nucleic acid encoding a Staphylococcus coagulase, hyaluronidase, mecA, or TSST polypeptide within a sample. The method comprises, or consists essentially of, (a) performing a nucleic acid amplification reaction using the sample as a source of template and a diagnostic device, wherein the device comprises a housing having a plurality of locations, wherein each of the locations contains a primer system, wherein the primers of each primer system are between 18 and 28 nucleotides in length and have a theoretical melting temperature between 58° C. and 62° C., wherein the device is capable of producing an amplification product diagnostic for nucleic acid encoding a Staphylococcus coagulase, hyaluronidase, mecA, or TSST polypeptide, and wherein each amplification product, when produced, is between 100 and 400 nucleotides in length, and (b) determining which locations of the device contain a primer system that resulted in the formation of amplification product, thereby detecting nucleic acid encoding a Staphylococcus coagulase, hyaluronidase, mecA, or TSST polypeptide. The sample can be a sample obtained from a human. The nucleic acid amplification reaction can comprise at least 10 cycles. The nucleic acid amplification reaction can comprise at least 20 cycles. The nucleic acid amplification reaction can comprise a denaturing step at about 94° C. or about 95° C. The nucleic acid amplification reaction can comprise an annealing step at about 60° C. The nucleic acid amplification reaction can comprise an extension step at about 72° C. The sample can be a mucus sample. The sample can be a sample obtained from the human using a swab. The sample can be a sample processed to obtain bacterial nucleic acid. Each of the locations can comprise an intercalating dye, wherein each amplification product, when produced, is labeled with the intercalating dye, and wherein determining which locations of the device contain a primer system that resulted in the formation of amplification product is based on a signal from the dye. The amplification reaction can be performed in a thermal cycler device configured to receive the diagnostic device. The determining step (b) can be performed in using a dye reader device configured to receive the diagnostic device. The amplification reaction and the determining step (b) can be performed in a machine configured to receive the diagnostic device, the machine comprising a thermal cycler device and a dye reader device. The machine can be capable of providing output indicating the presence of nucleic acid encoding a Staphylococcus coagulase, hyaluronidase, mecA, or TSST polypeptide. The machine can be capable of providing output indicating that the primer system detected the presence of nucleic acid encoding a Staphylococcus coagulase, hyaluronidase, mecA, or TSST polypeptide. The output can be a paper printout or a computer readable file.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIG. 1 is a top view of a microfluidic card.

### DETAILED DESCRIPTION

This document provides systems, devices, and methods involved in generating detectable polymers. For example, this document provides diagnostic systems, diagnostic devices, primer systems, and collections of primer systems. A diagnostic system can include a diagnostic device containing primer systems.

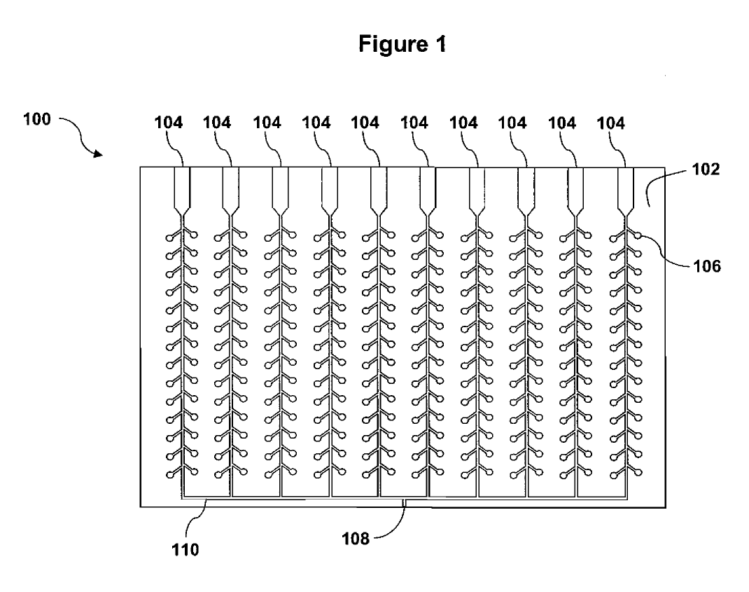

In general, a diagnostic device provided herein can include a housing having a plurality of locations. The housing can be any shape and size and can be made from any type of material including, without limitation, plastic, glass, silicone, or metal. For example, a housing provided herein can be rectangular, square, circular, or oval in shape, and can have a length, width, or diameter between five cm and 50 cm (e.g., between ten cm and 40 cm, between ten cm and 30 cm, or between ten cm and 25 cm). The depth or height of a housing provided herein can be between 0.2 cm and 2 cm (e.g., between 0.2 and 1 cm, between 0.3 and 1 cm, or between 0.5 and 1 cm). Each location of a housing can be configured to allow an amplification reaction to occur without primer system contamination from other locations. The locations of a housing provided herein can be any shape or size. For example, the locations of a housing provided herein can be in the configuration of a well or chamber with, for example, the ability to hold a volume between 1 μL and 100 μL (e.g., between 1 μL and 20 μL, between 1 μL and 10 μL, between 1 μL and 5 μL, between 10 μL and 50 μL, or between 15 μL and 25 μL). Such a volume can be 1.5 μmL, 10 μL, 20 μL, or 30 μL. In some cases, a housing can be a 96-well plate with each location being a well of the 96-well plate. A diagnostic device can be in the form of a microfluidic card. Such a card can have a series of locations and channels. The channels can provide fluid communication between a sample inlet port and one or more locations. For example, a housing can be a microfluidic card having one or more sample inlet ports in fluid communication with one or more locations via one or more channels. In some cases, such a housing can include one or more outlet ports for providing an outlet for added solutions or for providing an outlet for air so that fluid can flow through the channels. In one embodiment, a diagnostic device provided herein can be in the form of a microfluidic card with eight sample inlet ports each connected through channels (e.g., microcapillaries) to 48 locations (e.g., reaction chambers). Another example of a microfluidic card design is depicted in FIG. 1.

With reference to FIG. 1, microfluidic card 100 can have housing 102 defining a plurality of locations 106. While 280 separate locations are shown in this example, a housing provided herein can define any number of locations (e.g., 10, 25, 48, 96, 384, 1536, or more locations). Each location 106 can be in fluid communication with a sample inlet port 104 and an outlet port 108 via channel 110. Any number of channels can be defined by housing 102. For example, a housing provided herein can define one continuous, interconnected channel or can contain multiple separate channels.

A diagnostic device provided herein can contain a collection of primer systems and primer pairs. For example, each primer system or primer pair of a collection can be located at a different location defined by a housing so as to isolate each primer system or primer pair from other primer systems or primer pairs of a collection. For example, each primer system or primer pair of a collection can be housed within a separate location (e.g., a separate well of a plastic microtiter plate or a separate chamber of a microfluidic card). In some cases, each primer system or primer pair of a collection, or a subset of primer systems or primer pairs of a collection, can be housed together. For example, one primer system provided herein and one primer pair of a collection of 50 primer systems and primer pairs can be housed within a single well of a plastic microtiter plate with the remaining 48 primer systems and primer pairs being housed within separate wells. In some cases, a system or diagnostic device provided herein can contain at least one primer system set forth in Table 1 (e.g., at least two primer systems set forth in Table 1). In addition to containing any one or more of the primer systems set forth in Table 1 in any combination, a diagnostic device can contain primer systems not listed in Table 1. For example, a diagnostic device can contain a primer system similar to primer system number 1 with the exception that each nucleic acid primer is two nucleotides shorter than those of primer system number 1. In some cases, a diagnostic device can contain a primer pair designed to amplify host nucleic acid (e.g., human genomic nucleic acid or mRNA).

TABLE 1

Optimal primer systems for nucleic acid encoding a
Staphylococcus coagulase, hyaluronidase, mecA,
or TSST polypeptide (or portion thereof).

| | | | | | |
|---|---|---|---|---|---|
| Primer | | SEQ ID | | | |
| System No. | Primer Sequence | NO: | Length | Tm | Hits* |

Nucleic acid encoding Staphylococcal coagulase polypeptide

| | | | | | |
|---|---|---|---|---|---|
| 1 | CTTGAAATAAAACCACAAGGTACTGA | 1 | 26 | 59.88 | 28 |
| | ACTTCAATATCACTTGATTCTCCTTG | 2 | 26 | 59.16 | |
| 2 | AATGGAACAAAACAGACCATCTTTA | 3 | 25 | 60.15 | 6 |
| | TAAAATAGGGTTCGTTGGTGTACTT | 4 | 25 | 59.33 | |
| 3 | AAAGGATTATAGTGGGAAATCACAAG | 5 | 26 | 60.03 | 4 |
| | TCTAGCAGGTATTGGTCTTCCTCTA | 6 | 25 | 59.83 | |
| 4 | TCAAGGAGAATCAAGTGATATTGAG | 7 | 25 | 58.84 | 4 |
| | ATAATGTGATGCTTCTGTTGTTTCA | 8 | 25 | 59.95 | |
| 5 | CGCTAATGATTCTTGGAAAACTAAA | 9 | 25 | 60.04 | 1 |
| | AACGATTTCACCTTGTAACGTTTTAT | 10 | 26 | 60.40 | |

Nucleic acid encoding Staphylococcal hyaluronidase
polyeptide

| | | | | | |
|---|---|---|---|---|---|
| 6 | AATCGTTGTAACTGGATTCTTTGAT | 11 | 25 | 59.38 | 11 |
| | ATTGTCTTCTTAGGAACTGGCATTA | 12 | 25 | 59.61 | |
| 7 | TATTAAAACTTTCGCCCCAGATAGT | 13 | 25 | 60.55 | 2 |
| | GATGATCAATGTAAGAGCCATCTTTA | 14 | 26 | 59.90 | |

Nucleic acid encoding Staphylococcal mecA polypeptide

| | | | | | |
|---|---|---|---|---|---|
| 8 | GACAAGGTGAAATACTGATTAACCC | 15 | 25 | 59.26 | 77 |
| | TTCTAATGCGCTATAGATTGAAAGG | 16 | 25 | 60.14 | |
| 9 | TTATGTTGGTCCCATTAACTCTGA | 17 | 24 | 59.77 | 5 |
| | TACCTGAGCCATAATCATTTTTCAT | 18 | 25 | 60.11 | |
| 10 | TATTGTTAGCTGACTCAGGTTATGG | 19 | 25 | 58.83 | 4 |
| | GTCTCACCTTGTTTCATCTTGAGTT | 20 | 25 | 60.09 | |

Nucleic acid encoding Staphylococcal TSST polypeptide

| | | | | | |
|---|---|---|---|---|---|
| 11 | AATAATCAAAACTGCAAAAGCATCTA | 21 | 26 | 59.65 | 8 |
| | GTGTTTAAGTCAACTTTTTCCCCTT | 22 | 25 | 60.18 | |

*number of different gi numbers that is available in GenBank with nucleic acid sequences aligning with each primer of the indicated primer system.

The term “primer system” as used herein refers to a combination of two nucleic acid primers having the ability to amplify nucleic acid provided that the sequence of each nucleic acid primer is from 15 to 50 nucleotides in length and is such that it aligns without a mismatch to a coding region sequence, or its complement, for the indicated target polypeptide that is set forth in a GenBank® gi number listed in Table 2 or to a sequence, or its complement, that is within about 500 nucleotides (e.g., within about 450 nucleotides, within about 400 nucleotides, within about 350 nucleotides, within about 300 nucleotides, within about 250 nucleotides, within about 200 nucleotides, within about 150 nucleotides, within about 100 nucleotides, or within about 50 nucleotides) of a coding region sequence for the indicated target polypeptide that is set forth in a GenBank® gi number listed in Table 2. For example, each primer of a primer system provided herein can be from 15 to 45 nucleotides the length. In some cases, each primer of a primer system provided herein can range from 20 to 40 nucleotides (e.g., from 20 to 35 nucleotides, from 20 to 30 nucleotides, from 21 to 28 nucleotides, or from 23 to 27 nucleotides). The primer systems provided herein can be selected such that the length of amplified target nucleic acid, if present within an amplification reaction, would be between 100 and 400 nucleotides (e.g., between 150 and 350 nucleotides, between 175 and 325 nucleotides, or between 200 and 300 nucleotides). The theoretical melting temperature of each primer of a primer system provided herein can be between 58° C. and 62° C. (e.g., between 59° C. and 61° C., between 58° C. and 61° C., or between 58° C. and 62° C.). A primer's theoretical melting temperature is calculated as follows:

Tm=81.5+16.6(log 10([Na+]))+0.41*(% GC)−600/length

where [Na+] is 0.005 M. Each primer system provided herein can be used to amplify nucleic acid encoding a Staphylococcus coagulase, hyaluronidase, mecA, or TSST polypeptide (or a portion thereof) or nucleic acid that is within about 500 nucleotides (e.g., within about 450 nucleotides, within about 400 nucleotides, within about 350 nucleotides, within about 300 nucleotides, within about 250 nucleotides, within about 200 nucleotides, within about 150 nucleotides, within about 100 nucleotides, or within about 50 nucleotides) of nucleic acid encoding a Staphylococcus coagulase, hyaluronidase, mecA, or TSST polypeptide.

TABLE 2
Representative gi number for each primer system.

| Primer | | |
|---|---|---|
| System | | |
| No. | Target Polypeptide | GenBank ® gi number |
| 1 | Staphylococcus | 66390991; 66390996; 66390986; |
| | coagulase polypeptide | 66391006; 13624887; 13624891; |
| | | 13620261; 46736; 13624885; |
| | | and 13620259 |
| 2 | Staphylococcus | 13620237; 66391011; 13620253; |
| | coagulase polypeptide | 13620247; 13620245; and 915307 |
| 3 | Staphylococcus | 13620237; 13620263; 49243355; |
| | coagulase polypeptide | 47118312; and 13620239 |
| 4 | Staphylococcus | 66391001; 13620241; 29169948; |
| | coagulase polypeptide | and 20338652 |
| 5 | Staphylococcus | 66390981 |
| | coagulase polypeptide | |
| 6 | Staphylococcus | 19747210; 57284222; 87201381; |
| | hyaluronidase polypeptide | 87125858; 705405; 47208328; |
| | | 47118324; 49240382; 49243355; |
| | | and 47118312 |
| 7 | Staphylococcus | 38857526 and 38857527 |
| | hyaluronidase polypeptide | |
| 8 | Staphylococcus | 77993063; 2791912; 77993051; |
| | mecA polypeptide | 2791983; 2791919; 78217214; |
| | | 67003789; 78217209; 78217224; |
| | | and 78217201 |
| 9 | Staphylococcus | 62361778; 60651034; 62361782; |
| | mecA polypeptide | 62361784; and 62361780 |
| 10 | Staphylococcus | 2791901; 60687391; 2073520; |
| | mecA polypeptide | and 2791906 |
| 11 | Staphylococcus | 153122; 47208328; 18266750; |
| | TSST polypeptide | 47118324; 20278872; 18535665; |
| | | 11094374; and 82655308 |

The primer systems provided herein can share unifying advantageous features. For example, each primer system provided herein can amplify nucleic acid encoding a Staphylococcus coagulase, hyaluronidase, mecA, or TSST polypeptide (or a portion thereof) or nucleic acid that is within about 500 nucleotides of nucleic acid encoding a Staphylococcus coagulase, hyaluronidase, mecA, or TSST polypeptide. In addition, primer systems provided herein can be selected such that the length of amplified Staphylococcal nucleic acid would be between 100 and 400 nucleotides. Moreover, the theoretical melting temperature of the primer systems provided herein can be uniformly between 58° C. and 62° C., and the length of each primer of the primer systems provided herein can range from 15 to 50 nucleotides (e.g., from 21 to 28 nucleotides). These unifying characteristics can contribute to the effective detection of nucleic acid encoding a Staphylococcus coagulase, hyaluronidase, mecA, or TSST polypeptide present within samples.

The primer systems listed in Table 1 can be used effectively to detect a large group of different nucleic acid molecules encoding a Staphylococcus coagulase, hyaluronidase, mecA, or TSST polypeptide (or a portion thereof). For example, primer system number 1 can have the ability to detect Staphylococcal nucleic acid sequences that encode a Staphylococcus coagulase polypeptide (or a portion thereof) and are associated with 28 different GenBank® gi numbers.

Any method can be used to make the primers of a primer system provided herein. For example, chemical synthesis techniques such as those described elsewhere (Beaucage and Caruthers, Tetrahedron Lett., 22:1859-62 (1981)) can be used. In addition, nucleic acid primers can be obtained from commercial vendors such as MWG Biotech, Invitrogen, and Operon.

Any method can be use to make a system or diagnostic device provided herein. For example, a diagnostic device provided herein can be made as follows. A 384-well master plate containing 125 μL of one or more primer systems in dionized water at a working concentration of 100 nmole/1 μL of each primer can be constructed. The master plate can be used as a template source, and 1 μL of each master plate well can be transferred to corresponding wells on a 384-well microfluidic card. Spotted reagents can be allowed to dry at room temperature before the final plastic laminate layer of the microfluidic card is attached.

The primer systems provided herein can be used separately or in combinations with other primer systems provided in Table 1 or other primer pairs. When making a combination, any two or more primer pairs or primer systems provided herein can be arranged into any combination.

The diagnostic devices and primer systems provided herein can be used to detect nucleic acid encoding a Staphylococcus coagulase, hyaluronidase, mecA, or TSST polypeptide present within samples. For example, a sample can be obtained from a human (or other animal such as a monkey) and used in an amplification reaction to determine whether or not Staphylococcal nucleic acid is present in the sample. In some cases, nucleic acid within about 500 nucleotides (e.g., within about 450 nucleotides, within about 400 nucleotides, within about 350 nucleotides, within about 300 nucleotides, within about 250 nucleotides, within about 200 nucleotides, within about 150 nucleotides, within about 100 nucleotides, or within about 50 nucleotides) of nucleic acid encoding a Staphylococcus coagulase, hyaluronidase, mecA, or TSST polypeptide (e.g., a promoter or untranslated region) can be used to detect the presence or absence of nucleic acid encoding a Staphylococcus coagulase, hyaluronidase, mecA, or TSST polypeptide present within a sample.

Any type of sample can be used including, without limitation, a biopsy (e.g., punch biopsy, aspiration biopsy, excision biopsy, needle biopsy, or shave biopsy), a tissue section, lymph fluid, mucus, blood, serum, and saliva samples. A sample can be obtained from a human or any other animal suspected to contain Staphylococcus bacteria (e.g., birds, pigs, and horses). In some cases, a sample can be obtained from a mammal (e.g., a human) using a swab (e.g., an OmniSwab; Whatman). The presence of an amplification product following an amplification reaction using, for example, a human's mucus sample and a primer system provided herein can indicate that that sample contains Staphylococcus bacteria and that that sample contains Staphylococcus bacteria having the detected nucleic acid (e.g., nucleic acid encoding a coagulase, hyaluronidase, mecA, or TSST polypeptide (or portion thereof)). In such a case, the human can be diagnosed as being infected with Staphylococcus bacteria containing nucleic acid encoding a particular coagulase, hyaluronidase, mecA, or TSST polypeptide.

Some sample types can be pre-processed to enhance sample quality. For example, a mucus sample can be treated with a mucolytic agent to liquefy mucus within a mucus sample. Samples can be processed to concentrate the nucleic acid and render it in a form to facilitate successful PCR reactions. This includes, but is not limited to, common methods to disrupt bilipid membranes, such as the use of detergents, digestion of protein complexes, such as the use of proteinase K, and reduction of polymerase inhibitors through the use of selective affinity columns. Commercial kits for purification of DNA, RNA, or total nucleic acid are readily available from, for example, Qiagen and Roche. In some cases, a sample can be processed using a Qiagen QIAmp Viral RNA Mini Kit.

Any type of amplification reaction can be used in conjunction with the primer systems set forth in Table 1 to detect nucleic acid encoding a Staphylococcus coagulase, hyaluronidase, mecA, or TSST polypeptide (or portion thereof) or nucleic acid within about 500 nucleotides of nucleic acid encoding a Staphylococcus coagulase, hyaluronidase, mecA, or TSST polypeptide. For example, common PCR reactions designed to amplify nucleic acid from DNA or RNA can be used. Detection of RNA can be accomplished by synthesizing cDNA from RNA sequence templates. cDNA synthesis can be accomplished using standard methods using, for example, RNA-dependant DNA polymerases, such as reverse transcriptase. Such reactions can be primed with random oligonucleotide sequences, such as random hexamers and octamers, or by sequence specific oligonucleotide primers, including the same primers used for the PCR reaction. The cDNA synthesis can be performed in a separate reaction vessel from the subsequent PCR reaction (commonly referred to as two-step rtPCR) or in the same reaction vessel as the PCR reaction (commonly referred to as single-step rtPCR).

Purified DNA and cDNA samples can be pooled and added to a PCR master mix containing water, salt buffers, magnesium ions, nucleotide monomers (dATP, dCTP, dGTP and dTTP), native or engineered Thermus aquaticus DNA-dependant DNA polymerase, and an intercalating dye, such as Sybr Green or LC Green. The master mix and sample can then be added to a single loading port of a microfluidic card and dispersed to all the reaction wells using centrifugation. The cards can then be scored to isolate and seal each reaction chamber prior to thermocycling. The cards can be individually thermocycled using commodity block thermocyclers or many cards thermocycled simultaneously using air- or water-based thermocyclers such as the BioOven or the H2OBIT, respectively.

Positive PCR amplification reactions can be detected during thermocycling for quantitative or qualitative analysis (real time PCR) or after completion of thermocycling (qualitative end-point PCR). Signals can be detected through fluorescence-channel emission of substrate bound intercalating dyes using commodity real-time PCR capable PCR platforms or by end-point reads using microplate scanner platforms. Both types of platforms can be used for melting-point analysis for validation of positive signals.

### EXAMPLE 1

### Detecting Nucleic Acid Encoding a Staphylococcus Coagulase, Hyaluronidase, mecA, or TSST Polypeptide (or a Portion Thereof)

Samples were collected by surface swabbing with dry cotton-tipped swabs (BD; Franklin Lakes, N.J.). Collection surfaces were chosen based on those thought to be frequently touched by hospital workers, patients, and the general population. These surfaces included key pads of hospital entry locks, automated teller machines, vending machines, cashier payment stations, and gasoline pumps. Swabbed surfaces also included elementary school door handles, grocery store shopping cart handles, and sink areas of public bathrooms. Nasal swabs from three adults and two children were also collected.

All swabs were streaked within six hours on BBL CHROMagar Staph aureus plates (BD; Franklin Lakes, N.J.), which are selective by growth and color morphology for Staphylococcus spp. and grown for 24 to 48 hours at 36±1° C. Individual colonies were then isolated and streaked again on BBL CHROMagar Staph aureus plates as well as BBL CHROMagar MRSA plates (BD; Franklin Lakes, N.J.), which contain the antibiotic cefotoxin and were allowed to grow for an additional 24 to 48 hours. Individual bacterial colonies were selected from the secondary plating for total nucleic acid sample preparation using MagAttract Virus Mini M48 magnetic bead system and BioSprint 15 semi-automated platform (Qiagen; Germantown, Md.). All nucleic acid preparations were re-suspended in 75 μL and stored at 4° C. until use.

Individual PCR reactions consisted of 1 μL of the nucleic acid preparation 0.3 μM of each primer, and a total volume of 20 μL of a final 1× concentration of QuantiTech Sybr Green PCR master mix (Qiagen; Germantown, Md.). Reactions were placed in either 8-well strip tubes or 48-well trays, and real-time PCR was performed on a MiniOpticon (BioRad; Hercules, Calif.) with the following conditions: 94° C. initial hot start for 15 minutes, 40 cycles of 95° C. melting for 15 seconds, 60° C. anneal for 30 seconds, and 72° C. extension for 30 seconds, followed by a melting curve analysis from 60° C. to 95° C. in 0.2° C. increments. Real-time Sybr channel signal reading occurred during cycling at 72° C. immediately after each extension step. All reactions were held at 10° C. immediately following melting curve analyses for up to 24 hours and were then stored at 4° C. 1× master mix without nucleic acid template or PCR primers were included as blanks for each PCR run. PCR reactions were considered positive only if the Sybr Green signal was determined to be above the signal background (threshold level) within the 40 amplification cycles and if the derivative of the melting curve revealed a single product peak. The threshold level was determined as recommended by the thermocycler manufacturer by first subtracting the signal of the blank from all sample signals, averaging the non-signaling background through cycles prior to the first sign of amplification, and extending by 10 standard deviations.

One or more positive PCR reactions from each primer system were prepared for nucleic acid sequencing by purification with the QiaQuick PCR purification kit (Qiagen; Germantown, Md.). All sequencing reactions were performed by a sequencing service (Davis Sequencing; Davis, Calif.) using Big Dye Terminator V3.0 sequencing chemistry (Applied Biosystems; Foster City, Calif.). Trace files were processed with Phred (Ewing et al., Genome Res., 8(3): 175-185 (1998)), and resulting sequence strings compared to the current GenBank nr reference sequence collection with a local installation of NCBI BLAST (Altschul et al., Nucleic Acids Res., 25:3389-3402 (1997)). Sequences were considered valid if they significantly and exclusively matched Staphylococcus aureus reference sequence across the majority of recovered sequence.

At least one primer system for each target resulted in at least one positive real-time PCR signal (Table 3). In each case where a positive PCR product that resulted in a sequencing reaction of sufficient quality, the resulting sequence string was a significant and specific match to the correct Staphylococcus aureus target sequence.

TABLE 3
PCR results.

| Primer | | | PCR | Validated |
|---|---|---|---|---|
| System | | Test- | Prod- | to |
| No. | Target | ed | uct | Sequence |
| 1 | Staphylococcal coagulase polypeptide | Yes | Yes | Yes |
| 2 | Staphylococcal coagulase polypeptide | Yes | Yes | Yes |
| 3 | Staphylococcal coagulase polypeptide | Yes | No | N/A |
| 4 | Staphylococcal coagulase polypeptide | Yes | No | N/A |
| 5 | Staphylococcal coagulase polypeptide | Yes | No | N/A |
| 6 | Staphylococcal hyaluronidase | Yes | Yes | Yes |
| | polypeptide | | | |
| 7 | Staphylococcal hyaluronidase | Yes | Yes | Yes |
| | polypeptide | | | |
| 8 | Staphylococcal mecA polypeptide | Yes | No | N/A |
| 9 | Staphylococcal mecA polypeptide | Yes | Yes | Yes |
| 10 | Staphylococcal mecA polypeptide | Yes | No | N/A |
| 11 | Staphylococcal TSST polypeptide | Yes | Yes | Yes |

### OTHER EMBODIMENTS

It is to be understood that while the invention has been described in conjunction with the detailed description thereof the foregoing description is intended to illustrate and not limit the scope of the invention, which is defined by the scope of the appended claims. Other aspects, advantages, and modifications are within the scope of the following claims.

What is claimed is:

1. A device comprising a housing having a plurality of locations, wherein each of said locations contains a primer system, wherein the primers of each primer system are between 18 and 28 nucleotides in length and have a theoretical melting temperature between 58° C. and 62° C., wherein said device comprises at least one primer system capable of producing an amplification product diagnostic for nucleic acid encoding a Staphylococcus coagulase, hyaluronidase, mecA, or TSST polypeptide, and wherein each amplification product, when produced, is between 100 and 400 nucleotides in length.

2. The device of claim 1, wherein each of said locations is a chamber.

3. The device of claim 1, wherein each of said locations is a well.

4. The device of claim 1, wherein the primers of each primer system are between 23 and 27 nucleotides in length.

5. The device of claim 1, wherein the primers of each primer system have a theoretical melting temperature between 59° C. and 61° C.

6. The device of claim 1, wherein said housing comprises additional locations, wherein each of said additional locations contains a primer pair.

7. The device of claim 6, wherein at least one of said additional locations comprises a primer pair capable of producing an amplification product from human nucleic acid.

8. The device of claim 1, wherein each of said locations comprises an intercalating dye, and wherein each amplification product, when produced, is labeled with said intercalating dye.

9. The device of claim 8, wherein said intercalating dye is a green fluorescent dye.

10. The device of claim 8, wherein said intercalating dye is SYBR Green, LC Green, or SYTO9.

11. The device of claim 1, wherein each amplification product, when produced, is between 100 and 300 nucleotides in length.

12. A method for detecting nucleic acid encoding a Staphylococcus coagulase, hyaluronidase, mecA, or TSST polypeptide within a sample, wherein said method comprises:
(a) performing a nucleic acid amplification reaction using said sample as a source of template and a diagnostic device, wherein said device comprises a housing having a plurality of locations, wherein each of said locations contains a primer system, wherein the primers of each primer system are between 18 and 28 nucleotides in length and have a theoretical melting temperature between 58° C. and 62° C., wherein said device is capable of producing an amplification product diagnostic for nucleic acid encoding a Staphylococcus coagulase, hyaluronidase, mecA, or TSST polypeptide, and wherein each amplification product, when produced, is between 100 and 400 nucleotides in length, and
(b) determining which locations of said device contain a primer system that resulted in the formation of amplification product, thereby detecting nucleic acid encoding a Staphylococcus coagulase, hyaluronidase, mecA, or TSST polypeptide.

13. The method of claim 12, wherein said sample is a mucus sample obtained from a human.

14. The method of claim 12, wherein each of said locations comprises an intercalating dye, wherein each amplification product, when produced, is labeled with said intercalating dye, and wherein determining which locations of said device contain a primer system that resulted in the formation of amplification product is based on a signal from said dye.

15. The method of claim 12, wherein said amplification reaction is performed in a thermal cycler device configured to receive said diagnostic device.

16. The method of claim 12, wherein said determining step (b) is performed in using a dye reader device configured to receive said diagnostic device.

17. The method of claim 12, wherein said amplification reaction and said determining step (b) are performed in a machine configured to receive said diagnostic device, said machine comprising a thermal cycler device and a dye reader device.

18. The method of claim 17, wherein said machine is capable of providing output indicating the presence of nucleic acid encoding a Staphylococcus coagulase, hyaluronidase, mecA, or TSST polypeptide.

19. The method of claim 17, wherein said machine is capable of providing output indicating the primer system that detected the presence of nucleic acid encoding a Staphylococcus coagulase, hyaluronidase, mecA, or TSST polypeptide.

20. The method of claim 19, wherein said output is a paper printout or a computer readable file.


SEQUENCE LISTING (22 sequences)

SEQ ID NO: 1 - DNA, 26 bp, Staphylococcus aureus
cttgaaataaaaccacaaggtactga

SEQ ID NO: 2 - DNA, 26 bp, Staphylococcus aureus
acttcaatatcacttgattctccttg

SEQ ID NO: 3 - DNA, 25 bp, Staphylococcus aureus
aatggaacaaaacagaccatcttta

SEQ ID NO: 4 - DNA, 25 bp, Staphylococcus aureus
taaaatagggttcgttggtgtactt

SEQ ID NO: 5 - DNA, 26 bp, Staphylococcus aureus
aaaggattatagtgggaaatcacaag

SEQ ID NO: 6 - DNA, 25 bp, Staphylococcus aureus
tctagcaggtattggtcttcctcta

SEQ ID NO: 7 - DNA, 25 bp, Staphylococcus aureus
tcaaggagaatcaagtgatattgag

SEQ ID NO: 8 - DNA, 25 bp, Staphylococcus aureus
ataatgtgatgcttctgttgtttca

SEQ ID NO: 9 - DNA, 25 bp, Staphylococcus aureus
cgctaatgattcttggaaaactaaa

SEQ ID NO: 10 - DNA, 26 bp, Staphylococcus aureus
aacgatttcaccttgtaacgttttat

SEQ ID NO: 11 - DNA, 25 bp, Staphylococcus aureus
aatcgttgtaactggattctttgat

SEQ ID NO: 12 - DNA, 25 bp, Staphylococcus aureus
attgtcttcttaggaactggcatta

SEQ ID NO: 13 - DNA, 25 bp, Staphylococcus aureus
tattaaaactttcgccccagatagt

SEQ ID NO: 14 - DNA, 26 bp, Staphylococcus aureus
gatgatcaatgtaagagccatcttta

SEQ ID NO: 15 - DNA, 25 bp, Staphylococcus species
gacaaggtgaaatactgattaaccc

SEQ ID NO: 16 - DNA, 25 bp, Staphylococcus species
ttctaatgcgctatagattgaaagg

SEQ ID NO: 17 - DNA, 24 bp, Staphylococcus epidermis
ttatgttggtcccattaactctga

SEQ ID NO: 18 - DNA, 25 bp, Staphylococcus epidermis
tacctgagccataatcatttttcat

SEQ ID NO: 19 - DNA, 25 bp, Staphylococcus sciuri
tattgttagctgactcaggttatgg

SEQ ID NO: 20 - DNA, 25 bp, Staphylococcus sciuri
gtctcaccttgtttcatcttgagtt

SEQ ID NO: 21 - DNA, 26 bp, Staphylococcus aureus
aataatcaaaactgcaaaagcatcta

SEQ ID NO: 22 - DNA, 25 bp, Staphylococcus aureus
gtgtttaagtcaactttttcccctt